(19) United States
(12) Patent Application Publication
(10) Pub. No.: US 2004/0030355 A1
(43) Pub. Date: Feb. 12, 2004

(54) Apparatus for electrotherapy and method for testing and operating same

(75) Inventors: Alfred Schiller, Aeugst a.A. (CH); Johann-Jakob Schmid, Rifferswil (CH); Albert Cansell, Wissembourg (FR)

Correspondence Address: SHOEMAKER AND MATTARE, LTD, SUITE 1203, 2001 JEFFERSON DAVIS HIGHWAY, ARLINGTON, VA, 22202, US

(73) Assignee: Schiller AG, Baar (CH)

(21) Appl. No.: 10/622,190

(22) Filed: Jul. 18, 2003

(30) Foreign Application Priority Data
Jul. 20, 2002 (EP) 02016184.0

(51) Int. Cl. A61N 1/39

(52) U.S. Cl. 607/5

(57) ABSTRACT

An apparatus for electrotherapy has a checking circuit. The checking circuit is used for checking the functions of the apparatus in a test routine or for requesting the status of components in the apparatus. The apparatus is provided with communication means which interact with the checking circuit and are used for transmitting data, for example the result of a test routine, to a receiver.

### BACKGROUND OF THE INVENTION

The invention relates to an apparatus for electrotherapy, particularly a portable defibrillator, and also to a method for testing and/or operating such an apparatus.

Electrotherapy for cardiac problems frequently involves the use of defibrillators. Hospitals or first-aid staff are equipped with such appliances as standard. The electronics in more modern appliances allow user guidance and the output of user instructions. It is therefore possible for such appliances to be used even by persons without specific training. Efforts are therefore made to place such easily useable defibrillators in locations with public access, for example in stations, public buildings or company buildings. This is intended to allow electrotherapy to be performed for a patient immediately, even if a member of first-aid staff is not yet present or has not yet been advised. However, a problem with such defibrillators is that the appliance is hardly ever used under normal circumstances. It is therefore uncertain whether the appliance is still operational when seriously required. By way of example, the power in the power source, typically a battery or a storage battery, might no longer be adequate. It would also be conceivable for technical faults to appear on account of corrosion, rodent attack or vibration. Such appliances therefore require inspection—in a similar manner to fire extinguishers, for example. Such inspections are involved, however. They also cannot be readily undertaken by staff with no technical knowledge.

It is already known practice to design defibrillators such that a test routine is automatically performed at regular intervals. The result of the test routine is displayed to the user visually or audibly. Such apparatuses are described, by way of example, in U.S. Pat. No. 6,016,059, U.S. Pat. No. 5,800,460, U.S. Pat. No. 5,899,925, U.S. Pat. No. 5,579,234 or U.S. Pat. No. 5,591,213. Although the self-test described therein can also be used, in principle, by staff with no technical knowledge to establish whether the appliance is operational, regular inspection is still necessary, which results in high maintenance costs.

Another drawback of such appliances placed in areas with public access is that vandalism could result in appliances becoming damaged or purloined, so that an appliance is no longer available when seriously required. Another drawback of known appliances is that, during an attempt at electrotherapy by medically untrained staff, it will frequently be forgotten to notify a member of first-aid staff or a doctor in parallel. Even if untrained staff are able to perform electrotherapy successfully, medically trained staff should be involved in all cases.

Another problem of such known appliances is also that the test routine is likewise not performed in the event of total failure of the appliance.

### SUMMARY OF THE INVENTION

It is therefore an object of the present invention to avoid the drawbacks of the known situation, in particular that is to say to provide an apparatus for electrotherapy which combines little complexity for maintenance and inspection with allowing safe operation of the apparatus at all times, and therefore ensures that electrotherapy can be performed reliably when seriously required.

The invention achieves these objects with an apparatus and method described below.

The inventive apparatus for electrotherapy is typically in the form of a portable defibrillator.

The defibrillator is provided, in a manner which is known per se, with electrodes, a power source and a preferably electronic inspection and control device. The apparatus is provided with a checking circuit, particularly with a test circuit. The test circuit is used, by way of example, for checking the functions of the apparatus in a test routine. Such test routines are likewise already known. In line with the invention, the apparatus has communication means for data transmission. The communication means are or can be coupled to the checking circuit. The communication means can be used to transmit results and/or the sequence of the test routine or other results determined by the checking circuit to a receiver. The receiver is typically a service center belonging to a manufacturer of such appliances or a company which is engaged to maintain the appliances. Alternatively, it is conceivable for the receiver to be an emergency doctor or a hospital. In particular cases, the receiver could also be a responsible person in the organization which operates the apparatus, for example a responsible doctor in a railway company or a works fire brigade.

It is conceivable for any known communication means to be used. Thus, the apparatus can be connected, by way of example, to the public telephone network by means of a telephone line. Preferably, the apparatus is provided with a GSM module which is used for wireless data transmission between the communication means and the receiver, in which case public telephone networks are likewise used. Alternatively, other communication means are conceivable, for example UMTS telephones or local radio networks.

In line with a first preferred embodiment of the invention, the test circuit is designed to perform the test routine on a regular basis, in particular cyclically. The test routine can be performed every two weeks, for example. Alternatively, it is conceivable for the test routine to be initiated earlier, for example if particular ambient circumstances are detected. In addition, it is alternatively also conceivable for the apparatus to be addressed from the central control station. By way of example, a test routine can be initiated as a result of being called from a central control station. In this connection, the central control station can initiate a test routine cyclically, for example. It is also conceivable for the test routine to be performed extraordinarily upon particular occurrences, for example in very cold temperatures.

Thus, by way of example, a low temperature over relatively long periods of time can result in the battery prematurely no longer having adequate capacity to perform electrotherapy. In the event of such ambient conditions, a test routine can be initiated prematurely. In the present case, on a regular basis means in accordance with a particular rule. Regular performance of a test routine is also understood to mean, by way of example, performance of the test routine at inconstant intervals of time, for example after randomly determined intervals of time. Similarly, performance of a test routine can be initiated on a regular basis when particular ambient parameters are satisfied.

In line with a first embodiment of the invention, the test circuit interacts with the communication means such that a confirmation is transmitted to the receiver after every test routine performed.

In this way, the receiver can check centrally whether the test routine has been performed in one appliance or in every appliance among a plurality of appliances. If confirmation of the performance of a test routine is missing, it must be assumed that there is a fundamental fault in the appliance. Similarly, it must be assumed that there is a fundamental fault in the appliance if the apparatus does not respond to a call from the central control station. In this case, a service engineer can be appointed to inspect the appliance. The check on the sequence of the test routine can naturally be performed automatically by the receiver. Transmission of confirmation of the performance of the test routine can be transmitted in the form of an SMS (Short Message) for example. If an SMS has not been received by the receiver within a predeterminable period of time, the receiver can automatically initiate certain actions, for example can warn responsible persons.

In line with another embodiment of the invention, the checking circuit, i.e. the test circuit, for example, is coupled to the communication means such that a warning is transmitted to the receiver upon discovery of an error in the test routine. At the same time as the warning, it is also possible to transmit an error log, in particular. This ensures that the receiver is informed about the problems in the appliance, on the one hand. On the other hand, the actual error log might infer the error source, and the service engineer can take along suitable spares.

In line with another preferred embodiment of the invention, the apparatus is provided with a loudspeaker and/or with a microphone. The loudspeaker and/or the microphone are coupled to the communication means. They can be used for transmitting speech. The communication means can thus also be used to assist medically untrained staff in performing the electrotherapy as a result of voice transmission. The voice link can be set up automatically, for example, as soon as the appliance is started up.

The apparatus can also be provided with means for detecting the status of an energy source. Typical energy sources used are batteries or storage batteries, which reduce their capacity in the course of their useful life even when the apparatus is not operated. In addition, the sequence of the test routine also requires a certain electrical power. The measuring means for detecting the status of the energy source are likewise coupled to the communication means. As soon as the status of the energy source is no longer adequate, the communication means can likewise be used to transmit a warning to the receiver. The measuring means can be inspected using the checking circuit. Alternatively, it is conceivable for the status of the energy source to be checked independently and for any errors to be transmitted independently.

In addition, the communication means can also be designed to receive data. This can also be used for remote maintenance, in particular. If the error log reveals software errors as the cause of error, these can likewise be eliminated by means of remote maintenance. In addition, it is also conceivable to load new software releases in the appliance using the communication means. To this end, the communication means can be coupled to the computer arrangement and/or to the checking circuit.

In line with another preferred embodiment of the invention, the apparatus can be in a form such that the communication means are used to transmit a confirmation to the receiver whenever the apparatus has been used. This can firstly be used to record statistical details relating to the frequency of use. Following use, such an appliance should always be maintained. The confirmation can thus result in a service engineer being appointed for maintenance. In addition, the apparatus can be set up such that an alarm notification is transmitted to the receiver or to a further, predeterminable second receiver when the apparatus has been started up. Although modern appliances can, as mentioned, be used by medically untrained staff, it is nevertheless important for a patient to be subsequently put into medical hands. It is therefore advantageous for, by way of example, a member of first-aid staff to be automatically warned when the apparatus is started up.

It is also conceivable for the inventive apparatus to be provided with fitting means for fixture to a wall, for example. In this case, the apparatus can have sensor means which detect the presence of the apparatus in the fitting means. In this case, the communication means can be in a form such that a warning is transmitted as soon as the apparatus is removed from the fitting means. In this way, it is possible to establish if the apparatus has been purloined, for example. It is also conceivable for the apparatus to be provided with a GPS module. A GPS module incorporated in the apparatus has various advantages. If the appliance has been purloined, the position of the appliance can be ascertained. If the appliance is in emergency use, it is possible to ascertain the location of use on the basis of the GPS module. In this way, first-aid staff can be directed to a specific destination. The GPS module can therefore typically be used when the apparatus is removed from the holder or when the apparatus is started up.

The GSM module can be arranged either in the apparatus or in the holder. Arranging the GSM module in the holder has the advantage that the GSM module can be powered by means of a power supply, so that no batteries are used in the apparatus.

The inventive method for testing and/or operating an apparatus for electrotherapy is preferably carried out using an apparatus as described above. In a first step of the method, a test routine is performed on a regular basis. It can be performed cyclically or on the basis of other rules. Performance of the test routine involves checking whether a predeterminable test criterion is satisfied. If the test criterion is satisfied, information is transmitted to a receiver using communication means contained in the apparatus. It is also conceivable for information to be transmitted if the test criterion is not satisfied.

To check the test criterion, it is possible to check, by way of example, whether one or more of the following conditions are satisfied:

has a test routine been executed?

has an error been found in the test routine?

is the battery status no longer adequate?

has the apparatus been used?/is the apparatus in use?

has the apparatus been removed from fitting means?

The various test criteria can be implemented with various degrees of frequency. It may thus be necessary, for example, to check the presence of the apparatus in a holder more frequently. Test routines need to be performed only approximately every two weeks, however. When checking whether the apparatus is being started up or has been removed, a warning should be given quickly. In this connection, it is therefore preferable for an alarm to be transmitted as soon as the user has operated an on-button and/or as soon as the apparatus is removed from the holder.

The inventive apparatus can be used within the context of an overall system which comprises a central management point and a plurality of such appliances for electrotherapy. The central recording point is designed to receive and to evaluate the data transmitted by all apparatuses.

### BRIEF DESCRIPTION OF THE DRAWINGS

The invention is explained in more detail below with reference to the figures and using exemplary embodiments. In the figures:

FIG. 1 is a schematic illustration of an inventive apparatus,

FIG. 2 is a schematic illustration of the various components of an inventive apparatus,

FIG. 3 is a schematic illustration of a holder for the inventive apparatus,

FIG. 4 is a flowchart for the operation of the inventive apparatus,

FIG. 5 is a schematic illustration of a system having a plurality of inventive apparatuses, and

FIG. 6 is a schematic illustration of an alternative embodiment of a holder for the inventive apparatus.

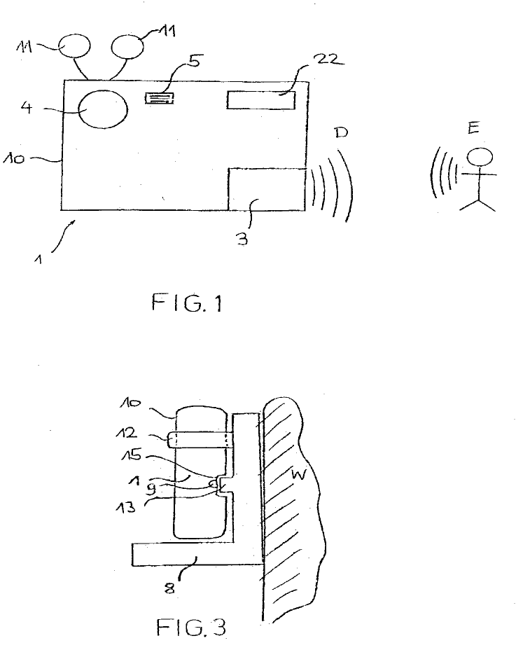

### DESCRIPTION OF THE PREFERRED EMBODIMENT(S)

Figure [lacuna] shows an apparatus 1 for performing electrotherapy. The apparatus 1 is typically in the form of a portable defibrillator. The apparatus 1 is provided, in a manner which is known per se, with electrodes 11 for sending electrical energy to a patient. The apparatus 1 also has further components of conventional defibrillators, which are not shown in detail.

The apparatus 1 is provided with a GSM module 3. The GSM module 3 is used for transmitting data D to a receiver E. The receiver E can be, by way of example, the manufacturer of the apparatus 1 or a company appointed to maintain it. Alternatively, the receiver E can be a hospital, a doctor, a member of first-aid staff or another organization, such as professional fire brigades or works fire brigades.

The appliance is also provided with a loudspeaker 4 and a microphone 5. The loudspeaker 4 and the microphone 5 are connected to the GSM module 3 in a manner which is not shown. The microphone 5 and the loudspeaker 4 allow voice communication between the user of the apparatus 1 and the receiver E. The apparatus is also provided with control elements 22. The control elements 22 typically comprise an on/off-button and means for initiating the therapy.

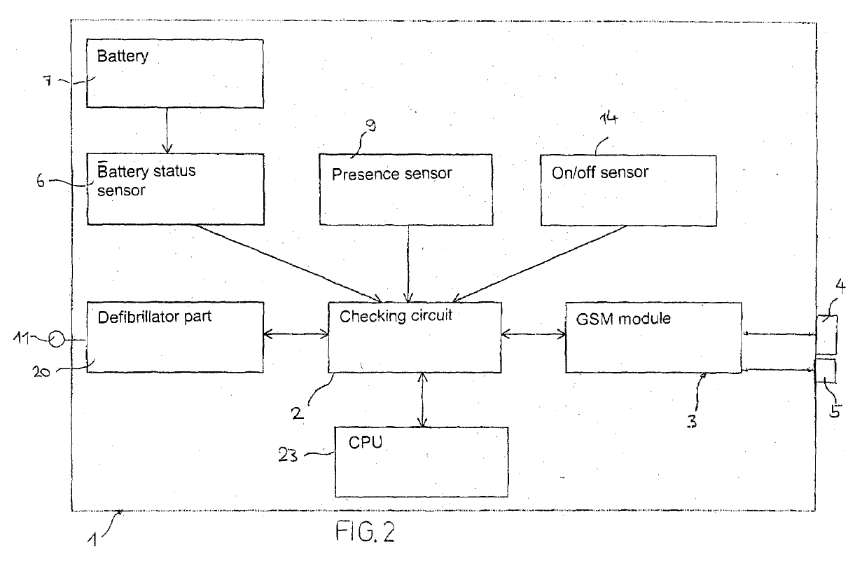

The apparatus 1 is arranged in a housing 10 which is sufficiently robust for use as a portable appliance. FIG. 2 shows components of the apparatus 1 which are fundamental within the context of the invention. The apparatus 1 is operated by a computer arrangement 23.

The computer arrangement 23 is a commercially available processor possibly provided with suitable memories. The apparatus 1 has a checking circuit 2, in particular. The checking circuit 2 is used for checking one or more test criteria. The checking circuit 2 is primarily used for regular, in particular cyclic, performance of a test routine for the defibrillator part 20. The defibrillator part 20 is of conventional design and has electrodes 11. The checking circuit 2 performs a test routine which is likewise essentially known and allows the fundamental parts of the defibrillator part 20 and of the processor 23 to be checked. The invention allows, but does not necessarily require, electrical energy to be output via the electrodes 11.

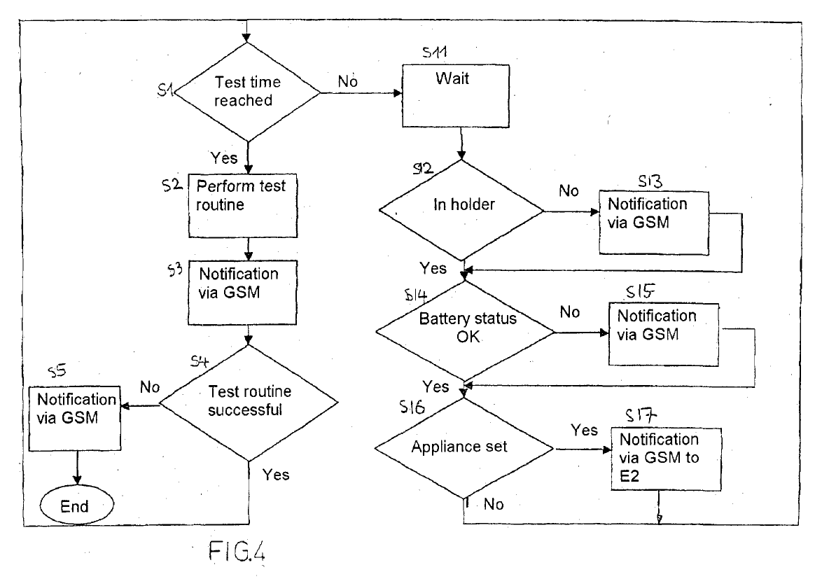

The checking circuit 2 can also be provided for checking various other criteria. The apparatus 1 is typically operated with a battery 7. The checking circuit 2 uses a battery status sensor 6 to check whether the status of the battery is still sufficiently high. The checking circuit can also use a presence sensor 9 to check whether the apparatus 1 is still held in a holder 8 (see FIG. 3). In addition, the checking circuit can use an on/off sensor 14 to check whether the apparatus 1 is in operation.

As soon as the checking circuit has established that a particular test criterion is satisfied, a GSM module 3 is used to transmit corresponding information to the receiver E. It is also conceivable for certain information to be transmitted directly and without using the checking circuit. Thus, by way of example, a notification can be automatically transmitted using the GSM module 3 when the appliance is turned on.

Connected directly to the GSM module 3 are a loudspeaker 4 and a microphone 5. Regardless of the rest of the operation of the apparatus 1, the loudspeaker 4 and the microphone 5 are used, in a manner which is known per se, to transmit voice information to the receiver E.

FIG. 3 schematically shows the fixture of an inventive apparatus 1 to a wall W of a publicly accessible building, for example. The wall has a holder 8 into which the apparatus 1 can be placed. The apparatus 1 is held in the holder 8 by means of a fixing strut 12. The holder 8 has a projection 13 which mechanically actuates the presence sensor 9 in a cutout 15 in the housing 10. Alternatively, other presence sensors are conceivable, for example electrical contact or optical sensors. As soon as the apparatus 1 is removed from the holder 8, the presence sensor 9 is deactivated or activated. This allows corresponding information to be transmitted to the receiver E or to another receiver using the GSM module 3.

FIG. 4 shows a typical sequence for the test method carried out using the inventive apparatus. In a first test step s1, a check is performed to determine whether a test time has been reached. To this end, the status of a clock (not shown) is requested in a manner which is known per se and is compared with a predetermined time condition. The test time entered can be, by way of example, a period of time of two weeks. If the test time has been reached, a test routine is performed in a step s2. The test routine tests the defibrillator part 20 in a manner which is known per se. As soon as the test routine has been performed, a notification is transmitted to the receiver E via the GSM module 3 in a step s3.

Following completion of the test routine, a check is performed in a step s4 to determine whether the test routine was successful. If the test routine was not successful, an error log is created and a notification is sent to the receiver E in step s5 using the GSM module 3. If appropriate, the error log (not shown) is likewise transmitted. When this information has been transmitted, the test sequence is complete. It must be assumed that the apparatus 1 is no longer operational. If appropriate, the receiver E can attempt to access the apparatus 1 by means of remote maintenance using the GSM module 3 and to perform repairs using software. If this succeeds, the test procedure is restarted and a fresh check is performed in step s1.

If the test time has not yet been reached in step s1, a predeterminable time is waited. When the time has elapsed in s11, a first checking step s12 involves a check to determine whether the apparatus 1 is in a holder 8. The waiting time in step s11 can be set to be relatively short. If the apparatus is no longer in the holder, a notification is transmitted using GSM in S13. In this case, the receiver E is informed that the apparatus has been removed from the holder 8. The battery status is then checked in s14. If the battery status is not OK, there is likewise a transmission to the receiver E2 in s15. In s16, there is then a check to determine whether the appliance is in operation. If the appliance has been set, information is transmitted to a further receiver E2 in s17. If the appliance has not been set, the sequence is restarted in s1.

It is also conceivable for the test criteria in s12, s14, s16 to be implemented independently of one another and at different times. In this case, the individual tests are advantageously, but not imperatively, performed and managed using the checking circuit 2 shown in FIG. 2. Typically, transmission of information to the receiver can be initiated when the apparatus is removed from the holder and when the appliance is turned on. It is then possible to dispense with the regular checking in s12 and s16.

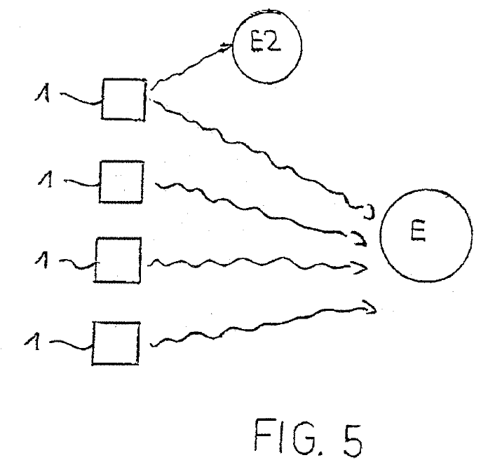

FIG. 5 schematically shows a system in line with the invention. The system comprises a plurality of apparatus 1 for electrotherapy which all communicate with a central receiver E. The individual apparatuses can also communicate with a further receiver E2 when specific test criteria, such as startup of the apparatus 1, has been satisfied.

The various appliances 1 are managed by the central receiver E. Typically, details about the age of the battery, the number of uses and errors are recorded and managed in a database. As soon as one of the appliances transmits information requiring inspection or repair of one of the appliances 1, the receiver E can react. This can involve either remote maintenance using the GSM module 3 or the employment of a service engineer.

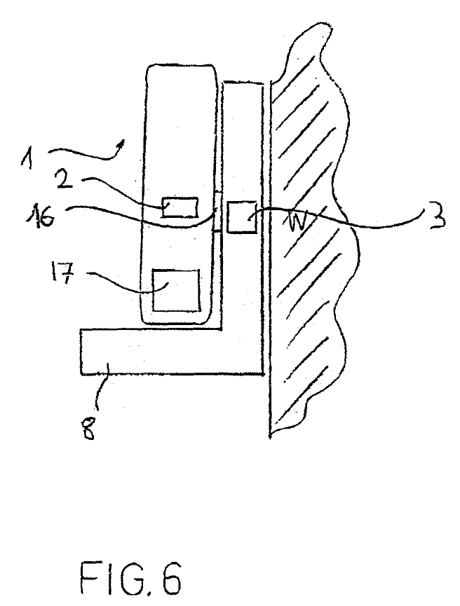

FIG. 6 shows an alternative exemplary embodiment of a holder 8. The holder is of similar design to the holder shown in FIG. 3. Unlike in FIG. 3, however, the GSM module 3 is arranged in the holder 8 and not in the apparatus 1. An electrical connection 16 which is automatically created when the apparatus 1 is put into the holder 8 allows communication between the GSM module 3 and the apparatus 1, particularly the latter's test circuit 2.

The apparatus 1 also contains a GPS arrangement 17. The GPS arrangement 17 allows localization of the apparatus 1 when it has been removed from the holder 8. The GPS arrangement can automatically be started up as soon as the apparatus 1 is removed from the holder 8.

A GPS arrangement is also useful in an exemplary embodiment as shown in FIG. 3, where the GSM module 3 is arranged in the apparatus 1.

It is also conceivable for the position report not to be transmitted by the GPS arrangement until the apparatus 1 is addressed externally, for example by the central control station. By way of example, the central control station can send an SMS notification as a request to the apparatus 1. When a corresponding SMS notification is received, the GPS arrangement is started up and reports the position of the apparatus 1 back to the central control station, for example using SMS.

By way of example, the opportunity to receive SMS can also be activated only when the apparatus 1 is removed from the holder 8. This additionally allows the battery in the apparatus 1 to be saved. In addition, it is conceivable to suppress the GSM module's search for the nearest suitable GSM cell for as long as the apparatus 1 is in the holder 8. When the apparatus is in the holder 8, the GSM connection is always made using the same, nearest transmitter. This also allows battery consumption to be reduced.

What is claimed is:

1. An apparatus for electrotherapy such as a portable defibrillator, having a checking circuit, particularly a test circuit for checking functions of the apparatus in a test routine or for ascertaining values of a internal condition of the apparatus, the apparatus further comprises communication means for data transmissions coupled or adapted to be coupled to the checking circuit, that communication means being designed to transmit to a receiver data selected from the group consisting of data relating to the performance of a test, data relating to results of the test routines and data relating to the internal condition of the apparatus.

2. An apparatus for electrotherapy, such as a portable defibrillator, having a checking circuit, particularly a test circuit for checking functions of the apparatus in a test routine and/or for ascertaining internal conditions of the apparatus, the apparatus further comprising communication means for data transmission which are coupled or adapted to be coupled to the checking circuit, wherein the checking circuit is designed in such a way as to execute the test routine on the basis of a command transmitted from a central control station to the checking circuit through the communication means.

3. An apparatus according to claim 1, wherein the communication means are designed for wireless data transmission.

4. An apparatus according to claim 3, wherein the communication means comprise a GSM module.

5. An apparatus according to claim 1, wherein the checking circuit is designed to perform the test routine on a regular basis.

6. An apparatus according to claim 5, wherein the checking circuit is designed to perform the test routine cyclically.

7. An apparatus according to claim 5, wherein the checking circuit interacts with the communication means such that a confirmation is transmitted to the receiver after each test routine performed.

8. An apparatus according to claim 1, wherein the test circuit interacts with the communication means such that the discovery of an error in the test routine prompts a warning to be transmitted to the receiver and, optionally, an error log to be transmitted.

9. An apparatus according to claim 1, wherein the apparatus comprises at least one loudspeaker and a microphone coupled to the communication means and used for transmitting speech.

10. An apparatus according to claim 7, wherein the apparatus is provided with measuring means for detecting the status of an energy source, and in that the measuring means for detecting the status of the energy source are coupled directly or indirectly to the communication means.

11. An apparatus according to claim 1, wherein the communication means are designed for receiving data, and in that the communication means are coupled or can be coupled, to at least one of a computer arrangement, checking circuit and to a defibrillator part.

12. An apparatus according to claim 1, wherein the apparatus is designed such that the communication means transmit a confirmation to the receiver each time the apparatus has been used.

13. An apparatus according to claim 1, wherein the apparatus is designed such that the communication means transmit an alarm to the receiver or to a predeterminable further receiver when the apparatus has been started up.

14. Apparatus according to claim 1, wherein the apparatus is provided with a GPS arrangement.

15. An apparatus according to claim 1, wherein the apparatus is provided with fitting means for fixing the apparatus, the apparatus being provided with sensor means detecting the presence of the apparatus in the fitting means, the communication means transmitting a warning as soon as the apparatus has been removed from the fitting means.

16. Apparatus according to claim 15, wherein the GSM module is contained in the fitting means.

17. A method for testing and/or operating an apparatus for electrotherapy according to claim 1, comprising the following steps:
performing a test routine is performed on a regular basis;
checking whether a predeterminable test criterion has been satisfied;
transmitting data to a receiver using communication means contained in the apparatus if the test criterion has been satisfied.

18. A method according to claim 17, wherein the test criterion is checked by checking whether at least one of the following conditions have been satisfied:
has a test routine been executed?
have errors been found in the test routine?
is the battery status no longer adequate?
is the apparatus being used?
has the apparatus been removed from fitting means?

19. A system with a plurality of apparatuses according to claim 1 and with a central recording point for receiving or for evaluating data transmitted by the communication means in the apparatuses to the central recording point.
